(19) United States
(12) Patent Application Publication
(10) Pub. No.: US 2026/0286400 A1
(43) Pub. Date: Sep. 24, 2026

(54) METHOD FOR PRODUCING TARGET PRODUCT FROM BIOMASS WASTE

(71) Applicant: SUZHOU XUNIVERSAL BIOTECHNOLOGY CO., LTD., Suzhou, Jiangsu (CN)

(72) Inventors: Zhenhao FANG, Suzhou, Jiangsu (CN); Christain Alberto PACHECO MARTINEZ, Suzhou, Jiangsu (CN); Laurynas VASIUKAS, Suzhou, Jiangsu (CN)

(21) Appl. No.: 18/881,077

(22) PCT Filed: Jun. 29, 2023
(86) PCT No.: PCT/CN2023/103777

§ 371 Date: Jan. 3, 2025

(30) Foreign Application Priority Data
Jul. 5, 2022 (CN) 202210789243.2

(51) Int. Cl. C12P 7/56; C07K 14/78; C12P 7/64

(52) U.S. Cl. CPC C12P 7/56; C07K 14/78; C12P 7/64

(57) ABSTRACT

A method for producing a target product from biomass waste is provided, including the following steps. (A) putting strain A into a first culture medium made from biomass waste for degradation and fermentation, inactivating reacted material, and isolating cell debris of the strain, wherein the strain A is an organic matter degrading strain; and (B) using the cell debris obtained in step (A) as a second culture medium, introducing a transgenic strain B into the second culture medium for heterologous gene expression, and isolating to obtain the target product.

### CROSS-REFERENCE TO RELATED APPLICATIONS

This application is a national stage of PCT Application No. Number PCT/CN2023/103777, having a filing date of Jun. 29, 2023, claiming priority to CN application No. 202210789243.2, having a filing date of Jul. 5, 2022, the entire both contents of which are hereby incorporated by reference.

### FIELD OF TECHNOLOGY

The following relates to a method of producing a target product based on biomass waste, specifically a method for producing lactic acid based on biomass waste.

### BACKGROUND

Biomass waste is a major source of solid waste, which mainly contains kitchen waste, feces, and plant straw (residual from tree pruning). Numerous environmental issues caused by a direct discharge of biomass waste have called for further development of biomass waste recycling and utilizing methods. The patent document CN102794294A reveals a process of treating biological waste as follows: separate dry and wet residential biological waste to obtain dry and wet waste; Dry garbage is sorted manually and then sent for recycling and reuse; Wet garbage is sent to the crusher for crushing treatment; Steam and crush the materials into a steaming machine to achieve sterilization and oil dissolution effects; The materials processed by pressing and steaming are sent to a press machine for pressing, resulting in residue and oil-water mixture; Separate the treatment of oil-water and slag. These methods, however, are characterized by complicated technological processes, low efficiency in waste disposal, and low production efficiency of target products, which cannot be compensated by the efficiency of biomass waste metabolic treatment and the acquisition of the desired products.

### SUMMARY

The purpose of the present disclosure is to provide a new production method for target products from biomass waste, where a mixed strain A is used to degrade biomass waste, and resulting metabolic cell debris is used as a culture medium for a single strain B to produce the target product, significantly metabolizing organic waste while efficiently expressing heterologous genes. The degradation and fermentation of metabolic biomass waste and the production reaction environment using host expression of heterologous genes are separated, and different strains are utilized to exert their characteristics, balancing the two goals of metabolizing organic solid waste and expressing heterologous genes to produce the target product.

To achieve these objectives, this disclosure adopts the following technical solution:

A method for producing target products based on biomass waste comprised of the following steps:
(A) Place strain A in the first culture medium made from biomass waste for degradation and fermentation. Strain A is an organic matter degrading strain, and the reacted material is deactivated to isolate the cell debris of the strain;
(B) Using the cell debris obtained in step (A) as the second culture medium, transgenic strain B is introduced for heterologous gene expression, resulting in the target product being obtained after the separation process.

In an embodiment, in step (A), the reacted material is deactivated and centrifuged, the supernatant is discarded, and the slurry material from the lower layer is taken as the second culture medium. Among them, the second culture medium is only composed of the aforementioned slurry-like substance, without adding any other substances. The slurry-like substance provides the necessary carbon and nitrogen sources for the growth of strain B, and optionally also provides trace elements, including but not limited to metal cations such as sodium and magnesium.

In an embodiment, strain A comprises a combination of one or more fungi and/or bacteria capable of metabolizing the biomass waste.

In an embodiment strain A comprises a combination of one or more fungi from the Trichoderma, Penicillium, Rhizopus, Trichoderma, Aspergillus, or Mucor genera.

In an embodiment, fungus A is an artificially domesticated fungus.

Furthermore, strain A can include a combination of one or more of Aspergillus oryzae, Rhizopus oligogenes, and Aspergillus niger.

In an embodiment, the source of biomass waste comprises a combination of one or more kitchen waste, feces, and plant straw.

In an embodiment, the biomass waste comprises a combination of one or more proteins, lipids, and carbohydrates.

In an embodiment, the target product should be comprised of proteins, lipids, or organic acids, and the heterologous gene transferred from strain B to the host cell must be selected from genes expressing proteins, lipids, or organic acids.

In an embodiment, the target product is comprised of lactic acid, bio-oil, or elastin collagen.

In an embodiment, the target product is lactic acid, and strain B is Rhizopus oryzae transformed with a lactate dehydrogenase gene. This lactic acid is used as a raw material for producing polylactic acid plastic masterbatch.

In an embodiment, in step (A), the strain A and the first culture medium are degraded and fermented in the first reactor, and the degraded fermented material is inactivated and centrifuged for separation; A water-insoluble product is obtained and used as the second culture medium.

In an embodiment, in step (B), strain B and the second culture medium undergo a reaction in a second reactor, and the resulting mixture is inactivated, centrifuged, and the separated water-soluble product is purified.

In an embodiment, the current method is implemented as follows:
Mix biomass waste and nitrogen source to form the first culture medium;
Introduce one or more strain from the Trichoderma, Penicillium, Rhizopus, Trichoderma, Aspergillus, or Mucor genera to the first culture medium for degradation and fermentation;
Perform high-temperature inactivation treatment on the products resulting from the degradation and fermentation mentioned above, centrifuge, discard the supernatant, and collect sludge from the lower layer;
Inject genetically modified strain B into the sludge and cultivate it;
Deactivate and isolate to obtain the target product.

In an embodiment, a method of producing lactic acid based on biomass waste is implemented as follows:
Mix biomass waste with nitrogen source to form the first culture medium, where salt ions could be added as well; the biomass waste could either include or not include starch;
Introduce one or more strain from Trichoderma, Penicillium, Rhizopus, Trichoderma, Aspergillus, and Mucor genera to the first culture medium for degradation and fermentation;
Perform high-temperature deactivation treatment on the products resulting from the degradation and fermentation mentioned above, centrifuge them, and discard the supernatant, keeping the sludge from the lower layer;
Inject genetically modified strain B to the sludge and ferment it for cultivation;
When the pH value drops to the set value, a neutralizing agent is added for the reaction to occur. After the reaction, centrifugation is performed, and the supernatant is taken. The supernatant is heated and esterified for hydrolysis to obtain lactic acid.
The neutralizing agent is (NH₂)₂CO—(NH₄)HCO₃—NH₃·H₂O or calcium hydroxide. The set pH value is less than 6.

The current disclosure adopts the formula mentioned above and has the following advantages compared to the prior applications:

In an embodiment, the method described in this disclosure adopts a two-stage fermentation reaction. In the first stage, strain A is first used to degrade and ferment biomass waste, ensuring efficient degradation and treatment of biomass waste and achieving successful treatment results. Then, the material resulting from fermentation gets deactivated, and the fungal/bacterial cell debris is used as the culture medium for the second fermentation reaction. Strain B (designed specifically for the target product, as a single strain that transfers heterologous genes expressing the target product) is introduced for efficient expression of heterologous genes to obtain the target product, such as lactic acid; Strain A and B respectively exert their characteristics, balancing the metabolism of organic solid waste and the expression of heterologous genes.

### BRIEF DESCRIPTION

Some of the embodiments will be described in detail, with references to the following Figures, wherein like designations denote like members, wherein:

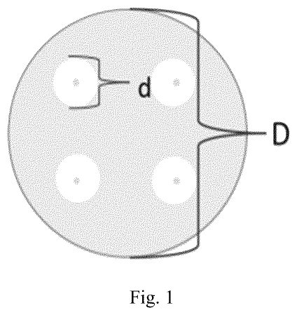

FIG. 1 shows a schematic diagram of a transparent circle on a simulated tablet;

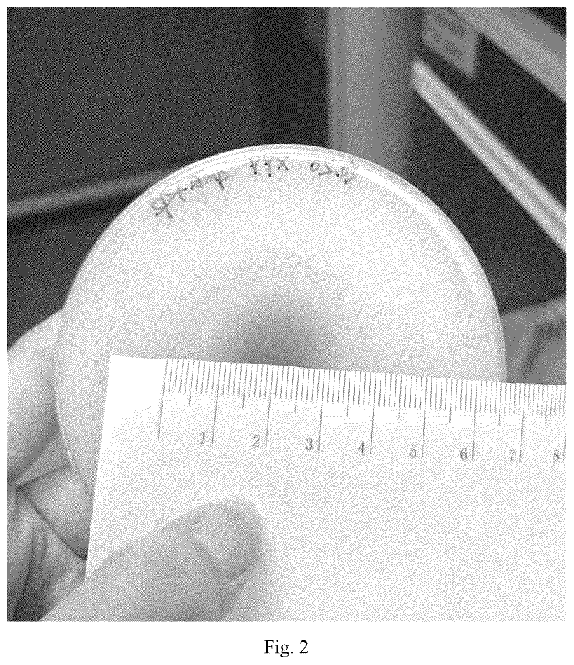

FIG. 2 shows a plate photo of protein degradation experiments by Aspergillus niger;

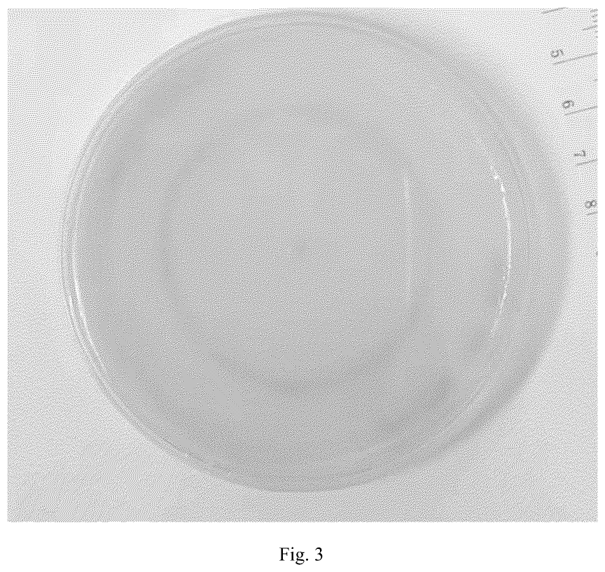

FIG. 3 shows a plate photo of lipid degradation experiments conducted by Aspergillus niger;

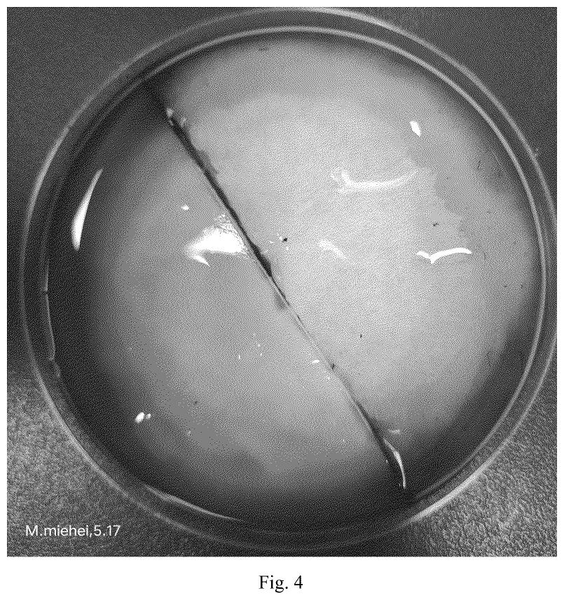

FIG. 4 shows a plate photo of starch degradation experiments conducted by Rhizopus oryzae;

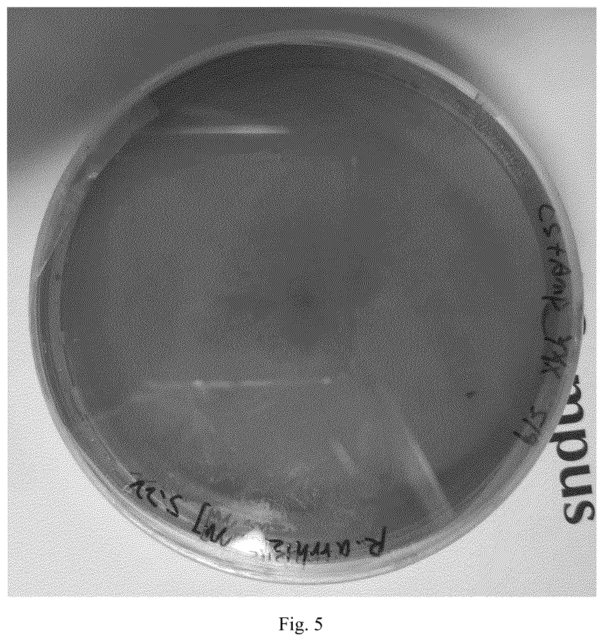

FIG. 5 shows a plate photo of cellulose degradation experiments conducted by Rhizopus oryzae;

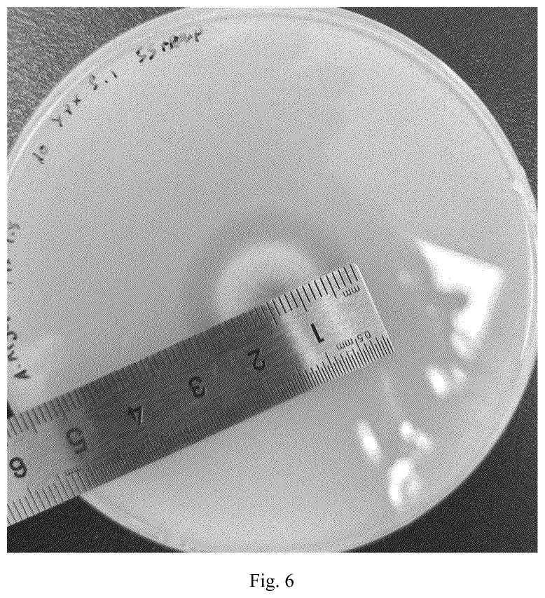

FIG. 6 shows a plate photo of starch degradation experiments by Aspergillus niger; and

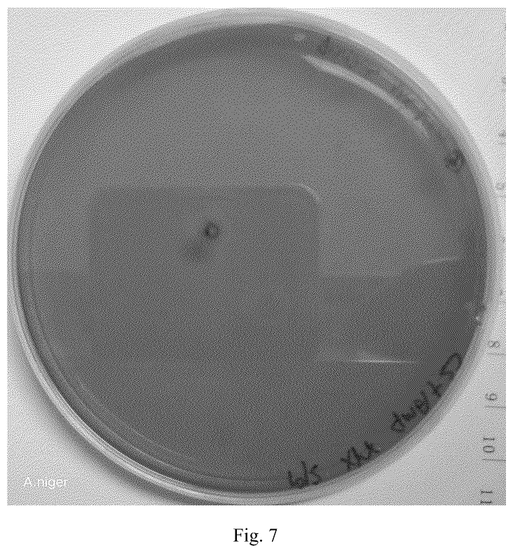

FIG. 7 shows a plate photo of cellulose degradation experiments by Aspergillus niger.

### DETAILED DESCRIPTION

In order to facilitate the understanding of the advantages and characteristics of this disclosure by those skilled in the art, the following chapter provides a detailed explanation of the embodiments of the present disclosure. It should be noted that the description of these embodiments is intended to assist in understanding the present disclosure, but does not constitute a limitation of the present disclosure.

To meet the needs of the Bihuan project, an efficient metabolism level of organic solid waste (kitchen waste) was achieved, and at the same time, the implanted heterologous genes were successfully expressed. The number of heterologous genes that each host cell can effectively carry is limited, and excessive implantation of heterologous genes may not necessarily kill the host, however, it can largely affect the host's survival ability. The present disclosure adopts a two-stage fermentation model, which separates the degradation fermentation of metabolic organic solid waste from the production reaction using host expression of heterologous genes. Different strains exert their characteristics, balancing the two goals of metabolic organic solid waste and the expression of heterologous genes. These two reaction environments are combined to form a closed production cycle.

(A) The first fermentation reaction consists of the following steps: add a slurry culture medium and strain A to the first bioreactor A, form carbon source of the slurry culture medium by crushing biomass waste (mainly kitchen waste, which may also include feces and plant residues), add nitrogen sources (such as agar, gelatin, etc.), or further add inorganic salts (such as sodium salts, etc.). Control the reaction conditions to enable strain A to metabolize biomass waste. Then, collect the reacted material from the first bioreactor A, and deactivate the entire reacted material (with the assistance of high-temperature and high-pressure), and then separate by high-speed centrifuge; The products dissolved in water (mostly organic compounds such as fungal protein residues) should be treated according to regulations; Water-insoluble products should be collected and used as the culture medium for the second stage reaction.

Moreover, strain A should be artificially domesticated or genetically modified in advance. Specifically, as follows:
101. Construction of heterologous plasmid for engineering strain A: this plasmid is used to enhance the parasitism characteristics of fungi/bacteria.
102. Screening of host cells of engineering strain A: the host cell selects fungi/bacteria with strong parasitism. Fungi are selected from Synchyprium, Achlya, Rhizopus, Trichoderma, Aspergillus, and Mucor; bacteria are selected from intestinal bacteria, including but not limited to Escherichia coli and methanogens.
103. Constructing Engineering Strain A: implant the constructed heterologous plasmid gene into the selected mixed strain. Specifically implanted through CaCl2/PEG method.
104. Cultivate engineering strain A, stimulate the growth activity of highly parasitic fungi/bacteria, expand the fungal/bacterial population, and then put it into the first bioreactor A.

In addition, strain A and slurry culture medium can also be continuously introduced into the first bioreactor A for continuous fermentation reaction.

(B) The second fermentation reaction includes the following steps:
201. Heterologous Telomere Construction of Engineering Strain B: Heterologous telomeres can be ldhA AmpRes telomeres.
202. Amplification of Telomere Volume: The volume of ldhA AmpRes telomeres was amplified by PCR and detected by Nanodrop Ultra Micro Instrument.
203. Constructing genetically engineering strain B: Implanting heterologous telomere genes into host cells. The host cell can be Rhizopus oryzae, and the lactate dehydrogenase gene can be implanted into Rhizopus oryzae using the CaCl2/PEG method to obtain transgenic Rhizopus oryzae.
204. Cultivate engineering strain B, stimulate the growth activity of highly parasitic fungi/bacteria, expand the fungal/bacterial population, and then put it into the second bioreactor B. Transgenic Rhizopus oryzae can be introduced into the second bioreactor B.
205. Use the fungal/bacterial cell debris resulting from the first reaction as the second culture medium and place it into the second bioreactor B. Then, introduce the engineered strain B (genetically modified Rhizopus oryzae) for fermentation reaction.

Engineering strain B and slurry culture medium can also be continuously introduced into the second bioreactor B for a continuous fermentation reaction.
206. Collect the reacted materials from the second bioreactor B.
207. Deactivate the entire reacted material by applying high temperature and pressure, then separate it via centrifugation; Collect the product dissolved in water (i.e. the supernatant after centrifugation, without cells) and purify it by esterification distillation to obtain the target product. The target product in this embodiment is lactic acid, which serves as a raw material for producing polylactic acid and can be further polymerized to produce a polylactic acid plastic masterbatch.

In step 205, when the pH level drops to the predetermined value, ammonia (such as (NH₂) 2CO—(NH₄)HCO₃—NH₃·H₂O) or calcium hydroxide is added to the second bioreactor B as a neutralizing agent, and the generated lactic acid reacts with the neutralizing agent.

In step 207, the esterification distillation purification mentioned above specifically includes: adding n-butanol to the water-soluble product and performing an esterification reaction between ammonium lactate and n-butanol. The generated n-butyl lactate is hydrolyzed to release the desired lactic acid.

It should also be noted that the heterologous gene expression range of engineering strain B is notably broad, encompassing the proteins, lipids, organic acids, etc., all of which can be used as raw materials for bioenergy or materials. However, the host cell may not necessarily be Rhizopus oryzae, and host fungal cells are mainly screened based on the target product; Engineering strain B uses parasitic strains to metabolize fungal remains in the first stage reaction. The main target products are raw materials for bioenergy and materials, such as lactic acid, bio-oil (biodiesel raw material), and elastic collagen (organoid raw material).

Generally, biomass waste, such as kitchen waste, is mainly composed of proteins, lipids, carbohydrates (such as starch), and cellulose. Thus, the degradation efficiency was calculated by simulating the garbage source on a culture medium plate and conducting transparent circle experiments. For a detailed description, please refer to Embodiment 1 listed below.

### Embodiment 1

### 1. Cultivation of Biowaste in a Petri Dish

A culture medium and a waste source simulation petri dish were made according to the instructions in Table 1 to simulate protein waste source, fat waste source, cellulose waste source, and starch waste source respectively. Circular plastic petri dishes with a diameter of 90 mm were used to conduct these experiments.

TABLE 1

| | |
|---|---|
| Protein waste source simulation | Non-fat powder milk (50 g/L) |
| petri dish, 250 ml | Agar (20 g/L) |
| Fat waste source simulation | Lard (120 mL/L) |
| petri dish, 250 ml | Agar 20 g/L |
| | (NH₄)₂SO₄ (2 g/L) |
| Cellulose waste source simulation | Congo Red (0.2 g/L) |
| plate, 250 ml | CMC-Na (7.52 g/L) |
| | Agar (16 g/L) |
| | Gluten (2 g/L 0 |
| Starch waste source simulation | Soluble starch (20 g/L) |
| petri dish, 250 ml | NaCl (5 g/L) |
| | Agar (20 g/L) |

### 2. Garbage Decomposition

The overall color of the garbage simulation petri dish was homogenous (such as white or red). After strain A decomposed protein, fat, starch, or cellulose, the color faded and formed one or several circles close to the agar color, as shown in FIG. 1. This experiment used circular plastic petri dishes with a diameter of 90 mm. The volume of each garbage simulation petri dish was 20 ml. The time for determining the diameter of the transparent circle was 72 hours after inoculation. Each part of the experiment utilized a standard concentration of 10 microliters from a self-made strain library. All strains were domesticated independently and put into the experiment. The calculation of the transparent circle and the calculation of petri dish component consumption are shown in the following equation:

$${( \frac{D}{2} )}^{2} : {( \frac{d}{2} )}^{2} = M : m$$

In this equation M represents the quality of the culture medium, m stands for the quality of the degraded culture medium, and the degradation amount of the target substance is obtained based on m and the concentration value in the culture medium.

### 3. Experimental Studies

3.1 Microcystis aeruginosa
(a) Experimental study on protein degradation by Microcystis aeruginosa

Following Table 1, a protein waste source simulation petri dish was made and water was added to skim milk powder, after which the entire petri dish turned white. 10 microliters of Aspergillus niger (concentration of 1×107 CFU) were inoculated into the petri dish containing the simulated protein waste and were cultivated at 35° C. for 72 hours. Due to the breakdown of protein by Microcystis aeruginosa, the white color faded and formed a circle on petri dish that was close to the color of agar. After all the measurements, the average circle diameter of the three parallel experiments comprised (3.32 cm+3.36 cm+3.12 cm)/3=3.27 cm. Protein consumption calculated according to the above formula accounted for 132 mg.

(b) Experimental Study on Lipid Degradation by Microcystis aeruginosa

A simulated fat waste source petri dish was prepared according to the instructions in Table 1. After adding water to the lard, the entire dish turned milky white. 10 microliters of Aspergillus niger (concentration 1× 107 CFU) were inoculated into the milky white petri dish containing the simulated fat waste and were cultivated at 35° C. for 72 hours. Due to the decomposition of fat by the Microcystis aeruginosa, the milky white color faded and formed a circle on petri dish that was close to the agar color. After all the measurements, the average circle diameter of the three parallel experiments comprised (5.01 cm+4.91 cm+5.11 cm)/3=5.01 cm. Lard consumption calculated according to the above formula accounted for 0.124 ml.

3.2 Rhizopus oryzae
(a) Experimental Study on Starch Degradation by Rhizopus oryzae

A starch waste source simulation petri dish was prepared according to the instructions in Table 1. Soluble starch was mixed with water, turning the whole substance white. Rhizopus oryzae (10 microliters, concentration 1×107 CFU) was inoculated into the petri dish containing the simulated starch waste and cultivated at 35° C. for 72 hours. As Rhizopus oryzae decomposed the starch, the white color faded and a circle close to agarose color was formed on the petri dish. In three parallel experiments, after 72 hours of cultivation, the substance in petri dishes became completely transparent and all starch was consumed, accounting for at least 400 mg of consumed protein.

(b) Experimental Study on Cellulose Degradation by Rhizopus oryzae

A cellulose waste source simulation petri dish was prepared according to the instructions in Table 1. Cellulose and Congo red were mixed with water to form a deep red color. Rhizopus oryzae (10 microliters, concentration 1×107 CFU) was inoculated into the petri dish containing simulated cellulose waste, and was incubated at 35° C. for 72 hours. As Rhizopus oryzae decomposed fat, the deep red color faded into light red, forming a light red circle on the petri dish. After all the measurements, the average circle diameter of the three parallel experiments comprised (3.32 cm+3.35 cm+3.09 cm)/3=3.25 cm. Cellulose consumption calculated according to the above formula accounted for 19 mg.

3.3 Aspergillus niger
(a) Experimental Study on Starch Degradation by Aspergillus niger

A starch waste source simulation petri dish was prepared according to the instructions in Table 1. Soluble starch was mixed with water which made the resulting substance appear white. Aspergillus niger (10 microliters, concentration 1×107 CFU) was inoculated into the plate containing the simulated starch waste and incubated at 35° C. for 72 hours. As Aspergillus niger decomposed starch, the white color faded and a circle resembling agar color was formed on the petri dish. After measurement, the average circle diameter of the three parallel experiments comprised (2.81 cm+2.62 cm+2.23 cm)/3=2.55 cm. Cellulose consumption calculated according to the above formula accounted for 12 mg.

(b) Experimental Study on Cellulose Degradation by Aspergillus niger

A cellulose waste source simulation petri dish was prepared according to the instructions in Table 1. Cellulose and Congo red were mixed with water to form a deep red color. Aspergillus niger (10 microliters, concentration 1×107 CFU) was inoculated into the deep red plate containing the simulated cellulose waste, and incubated at 35° C. for 72 hours. As Aspergillus niger decomposed fat, deep red color faded, forming a light red circle on the petri dish. After all the measurements, the average circle diameter of the three parallel experiments comprised (0.25 cm+0.32 cm+0.20 cm)/3=0.26 cm. Cellulose consumption calculated according to the above formula accounted for 0.0016 g.

### Embodiment 2

Step (1): A high-temperature of 121° C. and high-pressure of 0.12 MPa deactivation procedure was performed on each system in petri dishes resulting from each experiment in Embodiment 1. The resulting mixture was centrifuged, and the upper layer of water was discarded, while the slurry material from the lower layer was taken and mixed with the sludge collected after each experiment, inoculated Rhizopus oryzae transferred into ldhA AmpRes telomeres, while avoiding adding any other substances.

Step (2): The resulting mixture was cultivated at 32° C. for 72 hours, with a cultivation scale of 100 mL; When the pH value of the system dropped to 5.5, an (NH₂) 2CO—(NH4) HCO3-NH3·H2O neutralizer was added to the mixture; It was then centrifuged, filtered, and the supernatant was collected; The supernatant was then heated and n-butanol was added for esterification reaction to occur. Lactic acid production required the 1-hour application of 120° C. with a pressure of 270 mmHg. The final lactate production of three parallel experiments accounted for (1.680+1.600+1.427)/3=1.569 g.

### Embodiment Comparison

According to the instructions in Table 1, a culture medium containing protein, fat, and cellulose was prepared, with the rice root mold with ldhA AmpRes telomeres transferred into the medium and cultivated according to step (2) of Embodiment 2. During this stage, no lactic acid formation has been observed, and the content of protein, fat, and cellulose did not significantly decrease.

Embodiment 2 utilizes multiple strain capable of metabolizing biomass waste to convert biomass waste from various sources and components into a culture medium for genetically modified Rhizopus oryzae to produce lactic acid. The current method can be directly used to treat biomass waste with complex components (typically kitchen waste) and to produce lactic acid. As for starch-free biomass fertilizers, genetically modified Rhizopus oryzae rarely decomposes and produces very little lactic acid.

Any values appearing in this article are not limited to the exact range or value, and should be understood as those close to these ranges or values. As for numerical ranges, the final values and individual point values of each range can be combined with each other to obtain one or more numerical ranges, which should be considered as specifically disclosed in this article.

Unless stated otherwise, all technical and scientific terms used in this paper have the same meaning as generally understood by those skilled in the art. If there is any contradiction or inconsistency between the definition used in this article and the definition contained in other public literature, the definition used here shall prevail.

Unless explicitly stated in the context, the indefinite articles “1”, “one”, and “one type” located before an element or component in this article are meant to indicate the number of instances (i.e. occurrences) of the element or component in question. Therefore, “1”, “one”, and “one type” should be understood as including only one or at least one, while the singular form of the element or component also includes the plural form.

Although the present invention has been disclosed in the form of embodiments and variations thereon, it will be understood that numerous additional modifications and variations could be made thereto without departing from the scope of the invention.

For the sake of clarity, it is to be understood that the use of ‘a’ or ‘an’ throughout this application does not exclude a plurality, and ‘comprising’ does not exclude other steps or elements.

1. A method for producing a targeted product from biomass waste, the method comprising:
(A) putting strain A into a first culture medium made from biomass waste for degradation and fermentation, inactivating reacted material, and isolating cell debris of the strain, wherein the strain A is an organic matter degrading strain; and
(B) using the cell debris obtained in step (A) as a second culture medium, introducing a transgenic strain B into the second culture medium for heterologous gene expression, and isolating to obtain the target product.

2. The method for producing the target product from biomass waste according to claim 1, wherein in step (A), the reacted material is inactivated, centrifuged, Na supernatant is discarded, and she a lower layer of slurry material is taken as the second culture medium.

3. The method for producing the target product from biomass waste according to claim 1, wherein a fungus A comprises a combination of one or more fungi and/or bacteria capable of metabolizing the biomass waste.

4. The method for producing the target product from biomass waste according to claim 3, wherein the strain A comprises a combination of one or more of the genera Trichoderma, Penicillium, Rhizopus, Trichoderma, Aspergillus, and Mucor.

5. The method for producing the target product from biomass waste according to claim 3 wherein the strain A comprises a combination of one or more of Aspergillus niger, Rhizopus Oligogenes, and Aspergillus Niger.

6. The method for producing the target product from biomass waste according to claim 1, wherein a source of the biomass waste comprises a combination of one or more kitchen waste, feces, and plant straw.

7. The method for producing the target product based on biomass waste according to claim 1, wherein the biomass waste comprises a combination of one or more proteins, lipids, and carbohydrates.

8. The method for producing the target product from biomass waste according to claim 1, wherein the target product comprises proteins, lipids, or organic acids.

9. The method for producing the target product from biomass waste according to claim 1, wherein a heterologous gene transferred from the strain B to a host cell is selected from genes expressing proteins, lipids, or organic acids.

10. The method for producing the target product from biomass waste according to claim 1, wherein the target product comprises lactic acid, bio-oil, or elastic collagen.

11. The method for producing the target product from biomass waste according to claim 1 wherein the target product is lactic acid, and the strain B is Rhizopus oryzae transformed with a lactate dehydrogenase gene.

12. The method for producing the target product from biomass waste according to claim 1, wherein in step (A), the strain A and et e first culture medium are degraded and fermented in a first reactor, the degraded and fermented material is inactivated, and centrifuged for separation; obtaining a water-insoluble product and using the water-insoluble product as the second culture medium; in step (B), the strain B and the second culture medium react in a second reactor, and a resulting mixture is inactivated as a whole, centrifuged, and the separated water-soluble product is purified.

13. The method for producing the target product from biomass waste according to claim 1, is implemented as follows:
mix biomass waste and a nitrogen source to form the first culture medium;
introduce one or more strains from the Trichoderma, Penicillium, Rhizopus, Trichoderma, Aspergillus, and Mucor genera into the first culture medium for degradation and fermentation;
perform high-temperature deactivation treatment on products resulting from the degradation and fermentation processes, centrifuge the products, discard the supernatant, and keep a sludge from the lower layer;
add a genetically modified strain B to the sludge and cultivate; and
separate to obtain the target product.
